(19) United States
(12) Patent Application Publication
(10) Pub. No.: US 2004/0192638 A1
(43) Pub. Date: Sep. 30, 2004

(54) Method and composition for prolonging the residence time of drugs in the gut

(76) Inventors: John A. Oaks, Madison, WI (US); Paul Bass, Madison, WI (US); David M. Barnes, Oregon, WI (US); Noah P. Zimmerman, Gautier, MS (US)

Correspondence Address: Joseph T. Leone, DeWitt Ross & Stevens S.C., 8000 Excelsior Drive, Madison, WI, 53717, US

(21) Appl. No.: 10/819,701

(22) Filed: Apr. 7, 2004

Related U.S. Application Data
(63) Continuation-in-part of application No. 10/186,486, filed on Jul. 1, 2002.
(60) Provisional application No. 60/302,502, filed on Jul. 2, 2001.

(51) Int. Cl. A61K 31/7076

(52) U.S. Cl. 514/48

(57) ABSTRACT

The present invention provides methods and compositions for slowing the transit time of foodstuffs, pharmaceutical compounds, nutritional supplements, and vitamins through the gastrointestinal tract using cyclic GMP (cGMP) alone and cGMP in combination with succinate. The method and corresponding compositions prolong residence time of such compounds, and thereby increase absorption through the small intestine, increase bioavailability, and improve feed conversion ratios.

### CROSS-REFERENCE TO RELATED APPLICATIONS

This is a continuation-in-part of co-pending application Ser. No. 10/186,486, filed Jul. 2, 2002, which claims priority to provisional application Serial No. 60/302,502, filed Jul. 2, 2001, both of which are incorporated herein by reference.

### FEDERAL GOVERNMENT SUPPORT

This invention was made with United States government support awarded by the following agencies: NIH A143007. The United States has certain rights in this invention.

### BIBLIOGRAPHY

Complete bibliographical citations to the documents cited herein can be found in the Bibliography, immediately preceding the claims.

### FIELD OF THE INVENTION

The present invention generally concerns methods and compositions for prolonging the transit time of pharmaceutical compounds, vitamins, and nutritional supplements, “nutriceuticals,” and foodstuffs through the gastrointestinal tract of humans and other animals, thereby increasing absorption of those compounds. For purposes of the present invention and unless otherwise noted, the term “supplement” will refer collectively to pharmaceutical compounds, vitamins, nutritional supplements, nutriceuticals, drugs, and the like.

### BACKGROUND OF THE INVENTION

The gastrointestinal tract processes and absorbs food, as well as supplements. Compounds travel from the stomach, which stores and digests food and supplements, to the small intestine, which comprises three sections: the duodenum, the jejunum, and the ileum. The small intestine functions to absorb digested food and supplements.

The process of absorbing supplements and nutrients derived from food is controlled by a complex system of inhibitory and stimulatory motility mechanisms, which are set in motion when compounds are ingested. Specific receptors in the small intestine respond to the specific nutrients ingested and modulate the transit and absorption rate of compounds. The same factors that affect nutrient absorption influence the intestinal absorption of the supplements. The small intestine has the greatest capacity for absorption of these substances.

For absorption to proceed efficiently, the food and supplements must arrive at an absorbing surface in a form suitable for absorption and must remain there long enough and at a concentration that promotes absorption. The nutrients and supplements must then be absorbed by the mucosa of the intestine. Accordingly, considerable advantage would be obtained if the nutrients and supplements could be retained for a longer period of time within the small intestine for absorption to occur. The period of time during which compounds are in contact with the small intestine is crucial for the efficacy of absorption. Therefore, an alteration of motility rate that increases or prolongs transit time of ingested compounds will ensure optimal utilization of the absorptive surface.

Absorption of supplements in the small intestine is a function of the molecular structure and composition of the supplement itself, the small intestine's response to the supplement, and the overall transit time of the supplement through the small intestine. To the pharmaceutical industry, the residence time in the small intestine is of great significance because it affects the quantity of the drug absorbed. For example, in some cases only 1% of pharmaceutical compositions, even drugs for serious medical conditions, are absorbed by the intestine. If the transit time of the compound could be slowed, such that just 1% more were absorbed, the total drug absorbed would double, thereby improving therapeutic efficiency.

Several previous attempts to alter small intestinal transit times have either not been successful (Khosla and Davis, 1987; Davis et al. 1986) or have focused on malabsorption of fatty acids in patients with gastrointestinal conditions (for the purposes of nutrition and weight gain) (see U.S. Pat. Nos. 5,977,175 and 5,817,641). Attempts to alter intestinal motility patterns using tissue-invasive nematode parasites of rats show increased propulsion and decreased transit times (Castro, 1989). In contrast, lumen-dwelling, non-mucosal, non-tissue invasive organisms have been found to slow absorption and to alter motility in the gut of the host (Dwinell et al., 1998). However, no practical application of these findings for either of these parasites has been successfully adapted to improving drug, vitamin, nutrient, and nutritional supplement absorption in humans or animals. Thus, a need exists for specifically optimizing the bioavailability of ingested compounds by increasing residence times in the small intestine, thereby improving the overall efficacy of numerous pharmaceutical, supplemental, and nutritional compositions, as well as increasing the absorption of nutrients derived from foods.

The tapeworm Hymenolepis diminuta (H. diminuta) is a chronic parasite of the rat. The parasite resides within the lumen of the small intestine and migrates along the lumen in a diurnal fashion, corresponding to host food intake (Bråten and Hopkins, 1969; Read and Kilejian, 1969; Hopkins, 1970). Although H. diminuta secretes a number of small molecules, proteins and glycolipids (Pappas and Read, 1972a; Knowles and Oaks, 1979; Uglem and Just, 1983; Zavras and Roberts, 1985; Oaks and Holy, 1994), this tapeworm is not associated with obvious harmful effects to its rat host (Insler and Roberts, 1976). Some of these secretions regulate physiological processes of the tapeworm, such as growth (Cook and Roberts, 1991). Still other secretions inactivate host physiological processes, such as digestive enzyme activity (Pappas and Read, 1972a, b; Uglem and Just, 1983; Pappas and Uglem, 1990). Dwinell et al. (1998) postulated that a secretion from this tapeworm is capable of altering host enteric smooth muscle contractions.

In the uninfected rat and other vertebrate species, including humans, two patterns of electrical activity are present in the smooth muscle of the small intestine. Note that the electrical spiking of muscle cells is a reflection of muscle contraction, and hence an indirect measure of gut motility. The first pattern is the digestive pattern of myoelectric activity that occurs after nutrient ingestion and is characterized by random electrical spiking throughout the length of the small intestine. The second pattern of myoelectric activity, termed the Migrating Myoelectric Complex (MMC), is present in the interdigestive state (Szurszewski, 1969; Carlson et al., 1970). The MMC is divided into 3 phases: Phase I is a period of myoelectric quiescence, followed sequentially by Phase II, a period of irregular spiking activity, and Phase III, a period of maximum myoelectric spiking frequency and amplitude. Because Phase III migrates caudally along the small intestine and is a contraction closing off the intestinal lumen, it causes the propulsion of the lumenal contents to the colon. In the rat, a complete cycle of the MMC occurs approximately every 15 minutes. As a result, the MMC serves as the “housekeeper” of the small intestine, sweeping the remnants of the preceding meal, as well as any bacteria present in the lumen, toward the caecum and colon.

Parasitic infection can disrupt the MMC and induce a repertoire of myoelectric alterations characteristic of the specific parasite (Palmer et al., 1984; Berry et al., 1986; Dwinell et al., 1994; Palmer and Greenwood-Van Meerveld, 2001). In the case of the tapeworm, H. diminuta, there are two characteristic alterations of myoelectric activity: the Repetitive Burst of Action Potential (RBAP); and the Sustained Spike Potential (SSP). Homogenate fractions of whole tapeworms infused into the small intestinal lumen were shown to alter myoelectric activity by inducing RBAP and SSP indistinguishable from those induced by tapeworm infection (Dwinell et al., 1998). These RBAP and SSP alterations act to interrupt the normal myoelectric activity. By replacing the propulsive Phase III's with static RBAP and SSP, the normal orad to caudal propulsion of the lumenal contents is delayed. The result of the activation of these myoelectric patterns induced in the presence of the tapeworm is to slow movement of contents within the lumen of the intestine (Dwinell et al., 1997). This observation indicated that the physical presence of the tapeworm was not inducing altered myoelectric patterns (Dwinell et al., 1998), but some constituent present in the tapeworm was activating these myoelectric patterns in vivo. Culture medium, used previously to maintain H. diminuta in vitro, known as Tapeworm-Conditioned Medium (“TCM”), induces SSP, demonstrating that the tapeworm secretes compounds to its surroundings that cause changes in intestinal motility (Kroening et al., 2002).

### SUMMARY OF THE INVENTION

A first embodiment of the invention is a method for prolonging residence time of an administered substance in the small intestine of a subject. The method comprises administering to a subject in need of the substance a composition that comprises, in combination: cGMP, succinate, and a pharmaceutically suitable carrier. The composition is administered in an amount and form effective to promote contact of the cGMP and the succinate with the subject's small intestine. This has the effect of prolonging the residence time of the administered substance in the small intestine of the subject.

For purposes of the present invention and unless otherwise noted, the term “supplement” or “substance” refers collectively to pharmaceutical compounds, vitamins, nutritional supplements, nutriceuticals, drugs, and the like.

It is much preferred that the composition be administered orally in the form of a liquid solution containing from about 1.0 nM to about 100 mM cGMP and from about 1 mM to about 100 mM succinate (measured as the concentration of the free, non-protonated succinate di-anion). The composition can be administered concurrently with the substance (which is generally preferred), or administered prior to administering the substance, or after administering the substance (or any combination of before, during, and/or after). The composition may also be administered as a dry formulation using any of the conventional dry formulations well-known in the pharmaceutical arts (e.g., tablets, pills, capsules, powders, and the like).

The cGMP to be administered may be selected from (among other cGMP derivatives) cyclic guanosine 3′,5′-cyclic monophosphate; guanosine 3′,5′-monophosphate; 3′,5′-GMP; cGMP; guanosine 3′,5′-(hydrogen phosphate); guanosine 3′,5′-cyclic monophosphate; and guanosine 3′, 5′-cyclic phosphate. This list is exemplary only and non-limiting.

A second embodiment of the invention is the same as above, but is aimed at enhancing absorption of orally-administered pharmaceuticals, vitamins, and/or other supplements. Here, the method comprises administering to the subject a composition comprising, in combination, an absorption-enhancing of amount of cGMP and succinate, disposed in a pharmaceutically suitable carrier therefor.

A third embodiment of the invention is directed to a method of increasing bioavailability of an orally-ingested pharmaceutical, vitamin, or nutritional supplement. Here, the method comprises administering to a subject a composition comprising, in combination, a bioavailability-increasing amount of cGMP and succinate, disposed in a pharmaceutically suitable carrier therefor.

The invention also encompasses a composition of matter for prolonging residence time of an administered substance in the small intestine of a subject. The composition comprises, in combination, a residence time-increasing amount of cGMP and succinate, disposed in a pharmaceutically suitable carrier therefor. In the preferred formulation, the cGMP and the succinate are disposed in a liquid carrier having a cGMP concentration of from about 1 nM to about 100 nM and having a succinate concentration of from about 1 mM to about 100 mM (measured as the concentration of free succinate).

The instant invention also provides methods for use with livestock and other domesticated animals. As disclosed herein, the instant invention provides a composition and method for increasing the nutritive uptake of foodstuffs ingested by livestock. By administering the composition (per os) concomitant to feeding time, the passage of ingested food through the gut is slowed down, thereby allowing greater absorption of nutritive compounds from the feed. Administering cGMP orally as a means to increase nutritive uptake in monogastric animals is unknown in the prior art. The cGMP and/or succinate may also be administered to livestock to alleviate diarrhea in the animals.

The invention is also directed to a method of improving feed conversion ratios in monogastric animals. The method comprises administering to a monogastric animal a feed composition comprising a base feed ration in combination with an added amount of cGMP, wherein the added amount of cGMP is sufficient to improve the feed conversion ratio of the monogastric animal. The invention is further directed to a corresponding feed composition that comprises a base feed ration in combination with an added amount of cGMP, wherein the added amount of cGMP is sufficient to improve feed conversion ratios of monogastric animals fed the composition. The feed composition can also include an added amount of succinate.

Further advantages and uses of the invention will appear from a complete review of the Drawings and the Detailed Description, below.

### BRIEF DESCRIPTION OF THE DRAWINGS

FIG. 1 is an electromyogram recording depicting the myoelectric patterns of the small intestine of rats infused with saline (A) and cGMP (B) as described in Example 1.

FIG. 2 is a histogram illustrating the effect of lumenal cGMP dose on the induction of Sustained Spike Potentials (SSP) and the interruption of the orad to caudal myoelectric pattern illustrated in FIG. 1.

FIG. 3 is a histogram illustrating the effect of various substances infused into the intestinal lumen of rats on the frequency of SSP in Example 1.

FIG. 4 is a graph illustrating the increase in blood concentration of atenolol in the rat when administered intraduodenally with and without cGMP.

FIG. 5 is a histogram illustrating the effect of succinate in combination with cGMP on SSP frequency in rats.

### DETAILED DESCRIPTION OF THE INVENTION

The intra-cellular regulatory agent guanosine cyclic 3′, 5′-hydrogen phosphate (“cyclic GMP” or “cGMP”), a secretory product of the tapeworm H. diminuta, has been determined by the inventors to alter normal intestinal activity and thus slow the transit time of pharmaceutical compositions, vitamins, and nutritional supplements through the gut. As used herein, the term “cGMP” explicitly encompasses guanosine cyclic 3′, 5′-hydrogen phosphate, and other forms of cGMP, such as those listed in Budavari et al., Eds. (1980). Thus, as defined herein, the term “cGMP” explicitly includes, without limitation, cyclic guanosine 3′, 5′-cyclic monophosphate; guanosine 3′, 5′-monophosphate; 3′, 5′-GMP; cGMP; guanosine 3′, 5′-(hydrogen phosphate); guanosine 3′, 5′-cyclic monophosphate; and guanosine 3′, 5′-cyclic phosphate.

Additionally, the term “cGMP” as defined herein also includes any and all of the compounds that result from intestinal enzymatic alteration of cGMP, including: (1) the dephosphorylated ribonucleotide: riboguanosine or guanosine or deoxyriboguanosine or deoxyguanosine; (2) the other phosphorylated forms of cGMP: guanylate monophosphate or riboguanylate monophosphate or ribodeoxyguanylate monophosphate or deoxyriboguanylate monophosphate. These phosphorylated forms can occur as the 5′-monophosphate, the 2′-monophosphate, the 3′-monophosphate, and the 2′, 3′-monophosphate intermediate form; (3) the hydroxylated or deoxy forms of the ribose sugar of the nucleotide: ribose, deoxyribose, ribose monophosphate, or deoxyribose monophosphate; (4) the purine: guanine; (5) the methylated form of guanine: N2-methylguanine or N7-methylguanine; or (6) GMP's metabolic end products: xanthine and uric acid.

The term “succinate” as defined herein encompasses succinic acid (i.e., butanedioic acid), mono- and di-salts thereof, and mono- and diesters thereof. Thus, as defined herein, “succinate” includes, without limitation, succinic acid, monosodium succinate, disodium succinate, monopotassium succinate, dipotassium succinate, mono-C₁₋₆-alkyl succinates, di-C₁₋₆-alkyl succinates, and the like. A vast number of succinates are available commercially from a host of suppliers, including, for example, Aldrich Chemical Co. (Milwaukee, Wis.).

The pharmaceutical industry has published a great deal of information on the absorption times for pharmacologically-active agents and compounds. Such information is found in the numerous pharmacological publications, which are readily available to those skilled in the art. For example, if the in vitro model for absorption and release of an agent is 1.5 hours, then the small intestinal residence time for optimal absorption of the agent would be at least 1.5 hours. Thus, for pharmacologically-active agents, the appropriate residence time is dependent on the time for release of the active agent.

As a general rule, vitamins and nutritional supplements are absorbed in much the same way food molecules are absorbed, and times for absorption of these compounds should be similar to absorption times for foods containing similar vitamins and minerals. (There are, of course, exceptions to every rule. Most drugs move passively through the mucosal lining of the small intestine. However, certain complex molecules must undergo chemical processing (e.g., hydrolysis in the case of complex carbohydrates) prior to passing, or being carried, through the mucosa.)

As used herein, “digestion” encompasses the process of breaking down large molecules into their smaller component molecules, and “absorption” encompasses the transport, either active or passive, of a substance from the intestinal lumen through the barrier of the mucosal epithelial cells and into the blood and/or lymphatic systems.

In addition, the agricultural industry is very active in investigating ways of increasing the absorption (and hence the utilization) of nutrients, supplements, and drugs from foodstuffs in livestock. By providing a method to increase the absorption of nutrients and supplements already present in animal feed, the expense of buying feed is decreased and the nutritive state of the animals is increased. Thus, the efficiency of feed management (i.e., the feed utilization) in livestock is greatly increased.

Active Agent

To improve the efficacy of intestinal absorption of pharmaceutical agents, vitamins, nutriceuticals, nutritional supplements, and the like, residence time must be increased to enhance absorption. In short, absorption can be increased simply by increasing the amount of time a given supplement spends within the small intestine. The increased amount of time in transit through the gut allows for increased uptake of molecules from the gut, regardless of whether the uptake mechanism is active or passive. One means of increasing absorption of such supplements is to alter the contractility of the gut using the cellular regulatory agent cGMP, in combination with succinate. The current inventors have found that this particular combination of ingredients is quite efficacious to alter the motility of the gut. Advantageously, both cGMP and succinate are available commercially from a host of suppliers, such as Aldrich. As illustrated in the Examples below, cGMP combined with succinate significantly alters the motility of the gut, thereby increasing the residence time of substances therein.

The active ingredients, cGMP and succinate, may be compounded with the usual nontoxic, pharmaceutically acceptable carriers, binders, excipients, extenders, fillers, flavorants, colorants, buffers, preservatives, etc., (liquid, solid, or semi-solid) for tablets, capsules, solutions, emulsions, suspensions, suppositories, and any other dosage form suitable for use and known to the art. These carriers, excipients, and the like may include any ingredient suitable for use in manufacturing preparations of pharmaceuticals, supplements, or vitamins in solid, semi-solid, or liquid form. In addition, emulsifying, auxiliary, stabilizing, thickening, and coloring agents may be used. For example, gum acacia, gum agar, sodium alginate, bentonite, lactose, and powdered cellulose can be used. This recitation is by way of example only, and non-limiting.

cGMP and succinate are included in the pharmaceutical composition in an amount sufficient to produce the desired effect of altering motility in the gut. Pharmaceutical, vitamin, or supplement compositions containing the cGMP and succinate may be in any form suitable for oral use, including lozenges, hard gelatin caplets, soft gelatin caplets, tablets, suspensions, emulsions, and the like. They may also be mixed with inactive materials, such as water, oils, paraffins, powders, granules, syrups, detergents, salts, suspensions, or with agents for emulsifying, stabilizing, buffering, preserving, coloring, disintegrating, solubilizing, flavoring, sweetening, and the like.

When added to feed rations, the preferred method to incorporate the cGMP and/or succinate into the feed is to dissolve the active ingredients in an aqueous solvent and spraying the solution upon the feed (see the Examples). The cGMP and/or succinate can also be formulated as part of the feed, as (for example) by milling or pelletizing the cGMP and/or succinate along with the base feed itself Milling and pelletizing is done in the conventional and well-known fashion.

The effective dosage depends on a number of factors, including type of supplement that is to be administered in conjunction with the composition according to the present invention, the age and weight of the recipient, and the general health of the recipient, and the species of the recipient. Generally, an effective dosage is an amount that is effective to slow GI transit to allow the supplement additional time to be absorbed. One of ordinary skill in the art will be able to readily determine the optimum dosage, the procedure of administration, and the number of doses per day. In use, the composition, encapsulated or not, is typically ingested orally either prior to or along with the supplement to promote increased time in the lumen for absorption of the supplement. However, the composition may be administered by any effective means.

It is preferred that the compositions be administered in the form of solutions wherein the concentration of cGMP is from about 1 nM to about 100 mM and the concentration of succinate is from about 1 mM to about 100 mM. The solution can be administered in a single dose, before, along with, or after ingesting the supplement. The solution can also be administered in a series of doses, again either before, along with, or after ingesting the supplement.

In one preferred embodiment of the present invention, the pharmaceutical, vitamin, or nutritional supplement article is enterically combined in a suitable form with the cGMP, succinate, and/or any inactive agents. The cGMP/succinate combination produces a prolonged transit time in the small intestine, and the active drug, vitamin, foodstuffs, or nutritional compound is thus present in the small intestine over a longer period of time, thus increasing its absorption and bioavailability.

The invention is highly useful to make supplements efficacious in smaller doses. The invention can also be used to improve feed utilization in livestock animals. The invention is also useful to slow the effects of dehydration due to diarrhea. By slowing involuntary peristaltic actions in the gut, the present invention allows for greater absorption of water and nutrients out of the lumen of the small intestine.

The invention is also highly useful in improving feed conversion ratios in monogastric animals. The feed conversion ratio is the ratio between the amount of food fed to an animal and the corresponding weight gain of the animal. For commercial livestock operations, feed conversion ratio is a critical measure of success because the cost of feed is the largest single cost item in the budget. Thus, any additive to the feed that provides an increased weight gain in the animals, without increasing the amount of feed consumed, has a profound and beneficial economic effect. Similarly, maintaining weight gain in livestock with less feed input is economically beneficial for livestock producers.

In poultry, for example, the effect improving the feed conversion ratio is profound. Broiler chickens produced for the commercial markets have a remarkably short development time—roughly six weeks from hatchery to slaughter house. In this brief span of time, the chicks consume a vast amount of feed as they grow from mere ounces to broiler weight. Improving the feed conversion ratio (i.e., lowering the feed conversion ratio) directly improves the bottom line profitability of these operations by decreasing the amount of feed required to bring the chicks to market weight.

### EXAMPLES

The following examples are included solely to aid in a more complete understanding of the subject invention. The examples do not limit the scope of the invention described herein in any fashion.

The Examples described demonstrate that H. diminuta secretes myoelectric (motility)-altering compounds into in vitro culture media, and that the primary active compound is cGMP. Further, the Examples demonstrate that succinate acts as a potentiator of the activity of cGMP. (Succinate administered alone does not alter motility as compared to saline controls.) Thus, the Examples demonstrate that cGMP affects intestinal motility when administered alone, and these effects are enhanced when the cGMP is administered in combination with succinate. The Examples also demonstrate that when the composition of the present invention is administered with another drug or pharmaceutical compound, the uptake of the compound (and subsequent concentration of the compound in the blood stream) are significantly enhanced.

### Example 1

Mediation of Intestinal Contractility

The aim of this Example was to evaluate cGMP as an endogenous substance involved in those afferent neuro-sensory pathways that mediate tapeworm-induced changes in small intestinal smooth muscle contractility. Exogenous intraduodenally-administered cGMP was found to mimic the SSP pattern generated by a tapeworm infection in the rat. These novel observations increased the understanding of how intralumenal signal molecules associated with strictly lumenal parasites interact with neuro-pathways in host regulatory systems to activate a repertoire of intestinal pathophysiological responses.

Outbred male rats (Sprague Dawley, Harlan Sprague Dawley, Inc., Indianapolis, Ind.) were housed singly and maintained on a 12-hour light/12-hour dark cycle. All rats used in the bioassay procedure were uninfected with tapeworms. The design and surgical implantation procedure of the intestinal extracellular bipolar electrodes have been described previously (Dwinell et al., 1994, 1997). Briefly, four bipolar electrodes were surgically sutured to the intestinal serosa of each rat according to the methods of Dwinell et at. (1994), which is incorporated herein by reference. Three electrodes (J1-J3) were implanted on the jejunum at 10 cm intervals, with the first electrode (J1) placed 10 cm caudal from the ligament of Treitz and proceeding caudally with J2 and J3. A fourth electrode (designated “I” because it was placed within the ileum) was placed 20 cm orad from the ileo-caecal junction. In addition, a cannula was implanted with one end residing in the lumen of the mid-duodenum, whereas the other end was exteriorized to allow infusion of test fractions.

All compounds tested were infused into the duodenum via the cannula. Compounds were delivered in 0.2 ml aliquots followed immediately by a 0.2 ml saline cannula rinse. These volumes were used in order to prevent muscle contraction due to stretch from larger bolus volumes. To observe the induction of SSP, intestinal myoelectric activity was recorded for 90 minutes following infusion.

Consistent with the protocol of Dwinell et al. (1994), intestinal myoelectric activity was not recorded for the first five days after implantation surgery. Control recordings were taken after this period to assure the return of normal myoelectric patterns of the MMC following the cessation of post-surgical ileus. Periodic “control” recordings were made with saline on the intervening days between the tests with tapeworm-conditioned medium (TCM) or its fractions.

For intragastric infusion, rats (n=4) were lightly sedated with Halothane in order to insert a gastric tube per os and infused with 0.3 ml of 10 mM cGMP in saline directly into the lumen of the stomach. Thirty minutes of control myoelectric recording were always performed before infusion of any substance. Five to seven minutes after infusion, the rats were reconnected to the recorder. Effects of handling and anesthesia were not evident on intestinal motility since on their reconnection to the recorder all rats showed normal intestinal myoelectric activity. This procedure was repeated with the same rats on a different day with 0.3 ml 100 mM cGMP (equal to 100× the minimal dose required to increase SSP frequency by infusion into the lumen of the small intestine) and on a separate day with 0.3 ml saline as a vehicle control. Post-oral dosing myoelectric recordings were 90 minutes in duration.

Tapeworm-Conditioned Media

Tapeworms, used for in vitro culture, were collected 20-40 days after infection by flushing rat small intestine with room temperature (22° C.) Krebs-Ringer's-Tris Maleate buffer (KRTM, pH 7.2). Tapeworms of this age were selected because Dwinell et al. (1994) demonstrated that maximum altered myoelectric activity did not occur until at least ten days after infection. All tapeworms used for in vitro culture were from 35 cysticercoid infections per rat and all tapeworms transferred to culture were visually intact. The tapeworms were rinsed twice in KRTM and then twice more in sterile Roswell Park Memorial Institute (RPM) 1640 medium (Fisher Scientific, Chicago, Ill.) before being placed in culture.

To obtain TCM, five tapeworms were put into 50 ml of sterile RPMI 1640 (pH 7.2) containing 25 mM N-[2-hydroxyethyl] piperazine-N′-[2-ethanesulfonic acid] (HEPES), 100 U/ml penicillin, and 0.1 mg/ml streptomycin (Sigma Co., St. Louis, Mo.). The culture flasks with loosened caps were placed in a static tissue culture incubator (Forma Scientific, Marietta, Ohio) at 37° C., 80% humidity, and 5% CO₂/air, and then cultured overnight (approximately 12 hours).

To collect TCM, tapeworms were removed from the culture flasks with a sterile hook. However, before removing the tapeworms from the culture flasks, the color of the neutral red pH indicator was checked to determine that the pH was not below 6.8 and all tapeworms were visually inspected to insure that they were motile and intact at the end of the culture period. During these experiments, no autolysis or broken tapeworms were observed, nor was the pH of the TCM below pH 6.8 after overnight in vitro culture.

To partially characterize the signal factor(s) responsible for altering myoelectric activity, TCM was processed before bioassay in the following ways:

1. Passed through an Amicon DIAFLOW Ultrafiltration PM10 membrane (Millipore Corp., Bedford, Mass.) at 4° C. under pressure to collect molecules smaller than 10,000 MW.

2. Frozen up to 6 months at −20° C.

3. Boiled (100° C. for 30 minutes).

4. Chloroform extracted.

5. Extracted by the methods of Folsch et al. (1957).

6. Proteinase K digested.

After filtration TCM remaining above the filter, as well as the filtered TCM, were collected. Treated TCM and control samples were bioassayed for their ability to initiate SSP or RBAP myoelectric activity. Samples bioassayed included the following:

1. Saline (labeled “Saline”).

2. Control RPMI 1640 medium incubated under conditions identical to the preparation of TCM (labeled “RPMI”).

3. TCM removed directly from culture immediately before bioassay (labeled “Fresh TCM”).

4. TCM frozen for 24 hours, then thawed (labeled “Frozen TC”).

5. TCM filtrate containing <10,000 MW molecules (labeled “Filtered TCM”).

6. TCM retained under pressure but not allowed to pass through the membrane filter (labeled “Retained TCM”).

7. An “add back” of 1/1 (v/v) Filtered TCM plus Retentate (labeled “Filt+Ret”).

TCM was subjected to lipid extraction for nonpolar lipids by mixing equal volumes of chloroform and TCM and centrifuging at 15,000× g for 5 minutes at 4° C. The chloroform layer was removed and extraction of the aqueous layer with chloroform repeated (labeled “Chloroform Ext.”). Additionally, both polar and nonpolar lipids were extracted after the methods of Folch et al. (1957) and reported by Cain et al. (1977). Briefly, equal volumes of TCM and chloroform/methanol (2:1) were mixed. The upper chloroform-containing phase was removed and discarded. An equal volume of chloroform/methanol (2:1) containing MgCl₂ was thoroughly mixed with the retained aqueous phase and centrifuged as before, and the aqueous phase (labeled “Folch Wash”) was removed for testing in the rat bioassay.

The TCM was also subjected to proteinase treatment. Proteinase K bound to agarose beads (5 mg/ml; Sigma) was prepared per the manufacturer's instructions. The Proteinase K-agarose was rinsed twice in 50 mM HEPES buffer (pH 7.4) and resuspended in 200 μl HEPES buffer (pH 7.4). Then 100 μl of this suspension were added to 900 μl of TCM at 37° C. for 2 hours or overnight. Proteinase K-treated TCM (labeled “Proteinase K”) was then bioassayed as described below. To determine if the signal factor could be denatured, TCM was boiled for 30 minutes (labeled “Boiled TCM”).

Mettrick (1971) noted that in H. diminuta infected-rats, the pH of the small intestine was lowered to 5.5. Because the altered pH environment of the infected intestinal lumen might induce the myoelectric alterations observed in the presence of the tapeworm, RPMI 1640 medium adjusted to both pH 7.4 or 5.5 was tested in the bioassay system.

In vivo intestinal myoelectric activity was recorded with a polygraph chart recorder (Grass Instruments, Quincy, Mass.). The myoelectric signal from each electrode was simultaneously recorded on paper and by a personal computer with an I/O board and WINDAQ software (Dataq Instruments, Akron, Ohio) connected to the polygraph recorder by an analog-to-digital converter (Dataq Instruments).

Food was removed on each recording day at 0800 hour to prevent animal feeding and the subsequent disruption of the interdigestive myoelectric pattern. Recording occurred between 1300 and 1900 hours. All recording sessions for each animal were at least 1.5 hour in duration, and all animals were recorded while awake and unrestrained. Vehicle (0.9% saline or in some cases RPMI 1640) was used as control for test substances.

The following compounds were infused individually at the concentrations indicated (the asterisk [*] indicates a growth regulating factor for tapeworms identified by Zavras and Roberts, 1984, 1985): *acetate (50 mM), adenosine 3′, 5′-cyclic phosphate (cAMP; 10 nM-100 mM),*D-glucosaminic acid (25 mM), guanine (100 nm-10 mM), guanine monophosphate (10 mM), guanosine (10 mM), *guanosine 3′, 5′-cyclic monophosphate (cGMP; 100 nM-100 mM), lactate (20 mM), and *succinate (100 mM).

The following compounds were infused as a group: alanine (22.45 mM), asparagine (430 nM), aspartic acid (150 nM), glutamic acid (1.36 mM), glycine (1.33 mM), histidine (96.7 nM), isoleucine (381 nM), leucine (381 nM), lysine (274 nM), methionine (101 nM), phenylalanine (90.8 nM), proline (174 nM), serine (285 nM), threonine (168 nM), tyrosine (159 nM), and valine (171 nM).

All of the compounds recited in the two preceding paragraphs are compounds secreted by the tapeworm while in culture medium.

Two types of control recordings, a baseline and an intermittent control recording, were made with infusion of saline to determine if the electrodes were recording appropriately and to assure that the frequency of the normal interdigestive myoelectric patterns observed in these examples was consistent with those of previous studies (Dwinell et al., 1994, 1998). Five days after electrode and cannula implantation surgery, three consecutive baseline 90-minute recordings were made on separate days in each rat to confirm the presence of normal intestinal myoelectric activity at the end of the immediate post surgical period. In addition, intermittent control recordings were made in order to show that myoelectric activity remained normal over the course of the experimental period.

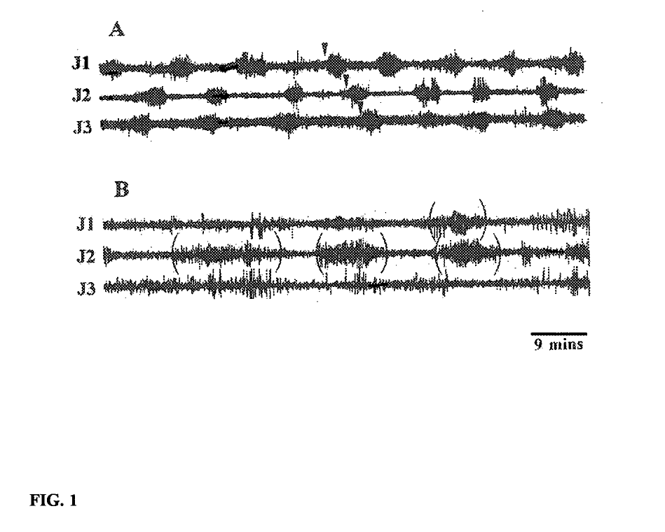

Secreted compounds were infused via the duodenal cannula and the frequency of the sustained spike potential (SSP) electrical patterns was determined from recordings of myoelectric activity. FIG. 1 illustrates the myoelectric patterns of the small intestine. The normal interdigestive myoelectric pattern on infusion of saline is shown in the recording of FIG. 1, Part A. Interdigestive patterns constitute the migrating myoelectric complex (MMC), a series of three phases marked by different levels of electrical spiking on the three electrodes, J1, J2 and J3. The third and final phase of the MMC (marked by arrowheads) is a period of >90% spiking and represents a series of contractions migrating orad to caudad along the small intestine. The caudad migration of phase III between electrode sites propels lumenal content toward the caecum.

The SSP on infusion of 10 mM cGMP are shown in the recording of FIG. 1, Part B. SSP are indicated by brackets on electrodes J1 and J2. SSP represent contractions that close the intestinal lumen, do not migrate from electrode to electrode and therefore do not propel intestinal contents. The frequency and lengths of the SSP and the reduction of phase III of the MMC frequency interact to reduce the overall propulsion of lumenal contents, resulting in a slowing of small intestinal transit.

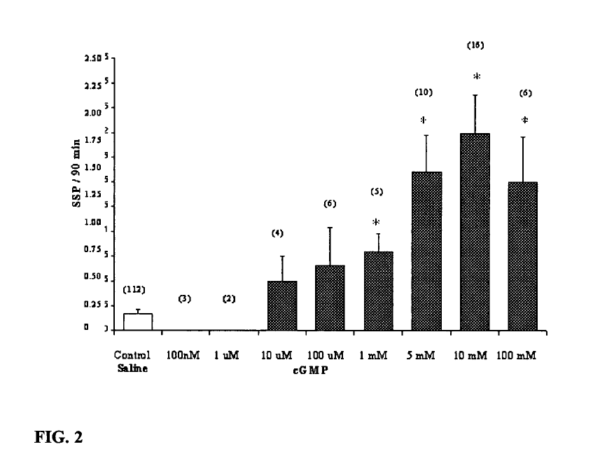

FIG. 2 illustrates the effect of lumenal cGMP dose on the induction of SSP. A significant increase in SSP frequency was seen in a range of 1-100 mM, indicating that SSP response to cGMP is dose dependent. The asterisk (*) indicates that the frequency of SSP is significantly different from the numbers of SSP occurring in response to saline. Numbers in parentheses are numbers of rats tested. Data were analyzed by the Student t-test. Significance was P≧0.05.

Of the substances tested, only cGMP initiated SSP myoelectric patterns. cGMP activated SSP in a concentration dependent manner. Both the TCM containing secreted cGMP and 10 mM cGMP in physiological saline directly infused into the intestine lose their ability to stimulate SSP when incubated with phosphodiesterase before bioassay.

Cyclic nucleotides were degraded to their 5′-monophosphate derivatives by incubation with bovine brain phosphodiesterase (PDE; cat. # P-0134, Sigma Co., St. Louis, Mo.). One activity unit of PDE (de-esterifies 1 μM cyclic nucleotide/min at 30° C.) was added to 1 ml of 10 mM cGMP or TCM and incubated at 30° C. for 16 hours. The samples were subsequently heated to 100° C. for 3 minutes to destroy PDE activity, allowed to cool to room temperature and then infused (0.2 ml aliquots followed immediately by a 0.2 ml saline cannula rinse) into the small intestinal lumen of uninfected instrumented rats via the duodenal cannula. A cGMP-specific ELISA (Amersham) determined that PDE treatment of both 10 mM cGMP and TCM reduced cGMP in both samples to below detectable limits. Recordings made after infusion of a test substance were 90 minutes in length. Control myoelectric recordings were taken both on the days before recording (90 minutes) and on the day of infusion (30 minutes) prior to sample infusion.

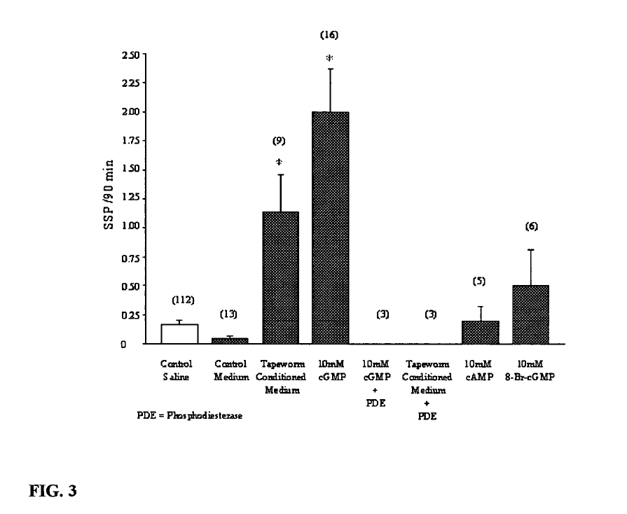

FIG. 3 illustrates the effect on the frequency of SSP of various substances infused (0.2 ml) into the intestinal lumen. Infusion into the intestine of cGMP (10 mM) and tapeworm conditioned medium collected 12 hours after incubations with 5 tapeworms significantly increased the frequency of SSP. Tapeworm condition from RPMI 1640 medium from 15-day-old tapeworms in 50 ml significantly increased the frequency of SSP as compared to the infusion of either control saline or control medium (RPMI 1640). In contrast, when 10 mM cGMP solution or TCM was incubated in phosphodiesterase (PDE) before infusion, control values for SSP frequency were also obtained. SSP frequency response to the infusion of 10 mM or 100 mM cAMP, 10 mM guanine, or 10 mM guanosine was not significantly different from saline controls. Table 1 below shows some of these results and the results of other tested substances.

TABLE 1

| | Number | SSP/Hr | p-value |
|---|---|---|---|
| TREATMENT | of Rats | (MEAN ± SE) | significance at <0.05 |
| Control (saline) | 112 | 0.17 ± 0.04 | |
| cAMP | | | |
| 10 nM | 1 | 0 | |
| 1 mM | 2 | 0 | |
| 10 mM | 5 | 0.13 ± 0.12 | 0.93 |
| 100 mM | 3 | 0 | |
| cGMP | | | |
| 100 nM | 3 | 0 | |
| 1 μM | 1 | 0 | |
| 10 μM | 4 | 0.5 ± 0.25 | 0.033 |
| 100 μM | 6 | 0.66 ± 0.38 | 0.29 |
| 1 mM | 5 | 0.80 ± 0.18 | 0.03* |
| 5 mM | 10 | 1.60 ± 0.38 | 0.005* |
| 10 mM | 16 | 2.0 ± 0.38 | 0.0003* |
| 100 mM | 6 | 1.50 ± 0.46 | 0.04* |
| 0.5 M | 4 | 1.25 ± 0.41 | 0.1 |
| 10 mM cGMP + 1 Unit PDE | 3 | 0 | |
| Guanine | | | |
| 0.1 mM | 1 | 1.33 | |
| 1.0 mM | 2 | 1.33 ± 0.94 | 0.53 |
| 10 mM | 2 | 0.33 ± 0.24 | 0.65 |
| Guanine suspension | | | |
| 10 mM | 3 | 0 | |
| Guanosine | | | |
| 10 mM | 4 | 0.50 ± 0.28 | 0.33 |
| Guanosine Monophosphate | | | |
| 10 mM | 6 | 0.5 ± 0.3 | 0.31 |
| Tapeworm secreted molecules: | | | |
| metabolic acids | | | |
| 10X Acid mixture | 4 | 1.67 ± 0.94 | 0.15 |
| 50 mM Acetate | 4 | 0 | |
| 20 mM Lactic Acid | 3 | 0 | |
| 100 mM Succinate | 3 | 0.33 ± 0.27 | 0.81 |
| 0.25 mM D-Glucosaminic Acid | 4 | 0 | |
| Tapeworm culture medium | | | |
| tapeworm culture medium + 1 Unit | 3 | 0 | |
| PDE | | | |
| Boiled tapeworm culture medium | 3 | 1.67 ± 0.27 | 0.04* |
| Boiled 10 mM cGMP | 3 | 1.0 ± 0.47 | 0.29 |
| 10 mM 8-Br-cGMP | 6 | 0.50 ± 0.31 | 0.38 |

None of the other tapeworm-secreted molecules, including the structurally related purine nucleotide, cAMP, were able to stimulate SSP above background levels as illustrated in FIG. 3. In addition, no response was observed on infusion of the cyclic nucleotides, cUMP and cIMP (data not shown). The cell-permeant cGMP analog, 8-Br-cGMP (0.2 ml of 10 mM), introduced into the intestinal lumen did not significantly increase the SSP frequency above background. These data suggest that the SSP myoelectric pattern is a specific response to the cyclized form of GMP and not a generalized response to purines or to other cyclized nucleotides. In addition, the lack of response to 8-Br-cGMP placed in the intestinal lumen strongly suggests that the receptor for cGMP is on the exterior of the cGMP-responsive cells in the intestine.

Neither cGMP (1.0 ml of 100 mM) injected intraperitoneally nor cGMP (0.3 ml of 10 mM and 100 mM) introduced into the stomach per os initiated the SSP pattern in the small intestine (data not shown). The lack of intestinal response to the infusion of cGMP into the stomach suggests that if gastric cGMP-responsive cells exist, they are not responsible for the SSP response in the intestine. The response of the intestine to lumenal infusion of cGMP but the lack of intestinal response to intraperitoneally-injected cGMP indicates that the receptors for cGMP are most likely on the luminal aspect of the small intestine. The lack of induction of SSP by other cyclic nucleotides strongly suggests that a specific cGMP receptor is involved. Taken together, these data indicate that cGMP secreted by Hymenolepis to the intestinal lumen can serve as a specific extracellular signal molecule regulating host small intestinal motility.

cGMP has been shown to activate the SSP, a unique myoelectric pattern in intestinal smooth muscle that closes the intestinal lumen for a relatively long duration (about 6.5 to about 45 min). The location for the cellular transducer of cGMP signaling in the host is unknown, but our data indicate that it is likely displayed on either lumenal epithelial cells or closely associated cells, such as the intrinsic or extrinsic neurons of the enteric nervous system. The evidence shows that slowed intestinal transit occurring as a result of tapeworm infection is the outcome of the cGMP-induced intestinal constriction (SSP) that diminishes transit.

### Example 2

Effect of cGMP on Drug Uptake

The purpose of this Example was to assess the rate of uptake of model drugs by the rat's small intestine treated with transit-slowing amounts of cGMP. cGMP was shown to be the signal molecule causing changes in the interdigestive smooth muscle contractile patterns and slowing transit in the lumen of the intestine. The contractile pattern caused by cGMP, i.e., SSP, did not migrate down the intestine and replaced propulsive contractile activity for up to 45 minutes. These patterns were originally observed in tapeworm infections of the rat, and tapeworm secretions were shown to contain cGMP.

Because uptake of compounds, e.g., drugs, nutrients, nutriceuticals, and the like, from the lumen of the intestine depends upon the length of exposure of these compounds to intestinal uptake mechanisms, the residence time of absorbed compounds in the intestine determines their exposure to uptake mechanisms. Slowing of the passage of compounds through the intestinal lumen increases the residence time for lumenal content, increasing the uptake of absorbed compounds and their subsequent bioavailability.

Thus, a model drug, atenolol, was introduced directly into the lumen of the rat's small intestine, and the concentration of atenolol was measured in the blood over time. The blood values and concentration kinetics of the model drug were compared after infusion into the intestine alone or when infused with cGMP. Atenolol was quantified using conventional liquid chromatography/mass spectrometry methods.

Outbred male rats (Sprague Dawley, Harlan Sprague Dawley, Inc., Indianapolis, Ind.) were housed singly and maintained on a 12-hour light/12-hour dark cycle. All rats used in this bioassay procedure were uninfected with tapeworm. For intestinal infusion, rats (n=2) were lightly sedated, and an indwelling cannula was implanted into the duodenal lumen in each of two rats. A second cannula was inserted into the superior vena cava near the cervical thoracic inlet and the rats allowed to recover for at least five days according to the protocol of Dwinell et al. (1994). In brief, the animal preparation involved the following: a cannula was surgically implanted into the duodenal region of each test rat. The cannula extended from the lumen of the duodenum, across the peritoneum, under the skin to the abdominal wall, and finally to an exit from the skin at the nape of the neck. A second cannula was installed at the nape of the neck and inserted into the superior vena cava near the cervical thoracic inlet. During the surgery, both cannulas were filled with sterile saline and plugged with metal pins. Rats were allowed at least five days to recover from implantation surgery before any manipulation occurred. All rats were housed individually after surgery to prevent damage to the cannulas. All experimental animals were eating and drinking freely during the five days before the experiment.

Following recovery, atenolol (1 mg/kg) was infused directly into the duodenal lumen. Blood samples were taken just before atenolol infusion (0 time) and 5, 10, 15, and 20 minutes after atenolol infusion. Both rats were first treated with atenolol alone.

Three days later, the experiment was repeated on the same rats, with the exception that a dose of 0.2 ml of 10 mM cGMP was administered via the duodenal cannula 15 minutes prior to infusing the atenolol, and 15 minutes after infusing the atenolol. The dose of atenolol was the same as in control experiment, 1 mg/kg.

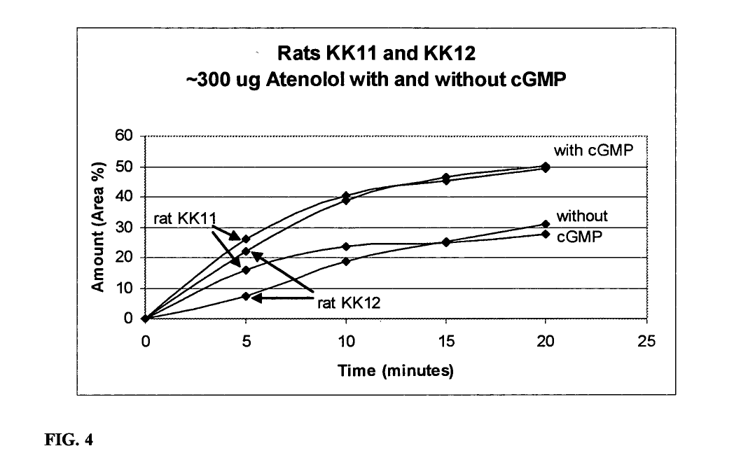

As illustrated in FIG. 4, treatment with cGMP almost doubles the amount of atenolol in the blood over the 20-minute experimental time period as compared to administration of atenolol to the same rats in the absence of added cGMP.

### Example 3

Effect of cGMP Combined With Succinate on SSP Frequency

In order to measure intestinal motility, rats were prepared as per Dwinell et al. (1994) as described in the prior Examples. Briefly, electrodes were implanted along the length of the intestine to allow observation of intestinal muscle contraction, and a cannula was implanted in the duodenum to permit the infusion of test substances. After normal intestinal motility was established in each rat, test substances were infused through the duodenal cannula, and myoelectric recordings were used to observe the occurrence of SSP. As noted earlier, SSP are prolonged contractions of the small intestine that do not migrate down the intestine and therefore act to slow the movement of substances, such as drugs, from stomach to large intestine.

Test substances included the following: 10 mM cGMP (known to induce SSP, see Example 1), 2.8 nM cGMP (the concentration secreted by the tapeworm; a concentration that is at or near the lower threshold of inducing SSP; Kroening et al., 2003), 12 mM succinate (the concentration secreted by the tapeworm), 100 mM succinate alone (approximately 9 times the concentration secreted by the tapeworm), 2.8 nM cGMP and 12 mM succinate together (both at concentrations secreted by the tapeworm), and 44.8 nM cAMP alone (the concentration secreted by the tapeworm).

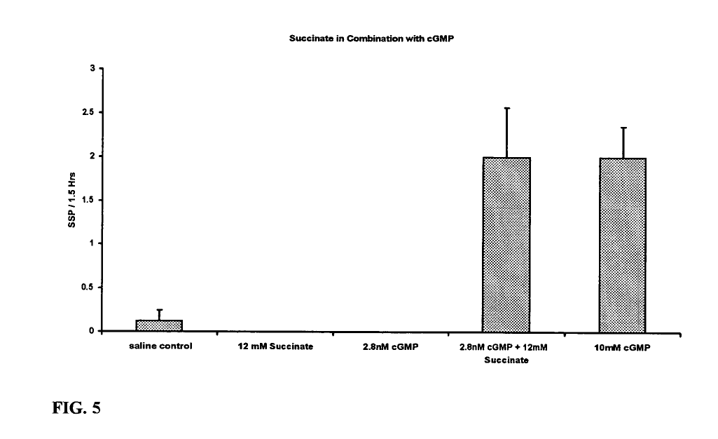

The results, illustrated in FIG. 5, show that infusing 2.8 nM of cGMP alone did not stimulate SSP above the background; however, when 2.8 mM of cGMP was infused in combination with succinate at a concentration of 12 mM, this combination was able to induce increased SSP frequency equal to that of 10 mM cGMP alone.

The data presented in this Example clearly indicate that cGMP results in induction of an SSP response and further that succinate intensifies this effect. The prolonged SSP response results in interruption of normal intestinal myoelectric patterns and decreased transit of compounds in the small intestine. Compounds infused in the gut during periods of prolonged SSP response have an increased blood concentration as compared to compounds not present during periods prolonged of SSP frequency, FIG. 4.

As shown in this Example, the SSP response can be initiated by co-infusion, at physiologic levels, of about 2.8 nM cGMP and 12 mM succinate. This was the lowest concentration tested, and thus it is quite likely that the combination is effective at lower doses of both cGMP and succinate. Thus, the invention disclosed herein provides compositions and methods allowing for a significant slowing of intestinal transit time and allowing for increased absorption of compounds and molecules from the small intestine.

Overall, the data illustrated in FIGS. 1 and 2, coupled with the findings of Dwinell, et al. (1997) (demonstrating that SSP slow transit), and further coupled with the atenolol uptake data generated in Example 2, the Examples clearly show that the decrease in intestinal transit arising from the increased SSP frequency allows increased absorption of atenolol (see FIG. 4). In short, the data taken as a whole indicate that increased atenolol absorption from the gut is a result of cGMP-induced SSP (and the concomitant increase in intestinal transit time) This resulted in a prolonged residence time for atenolol in the gut, thus allowing for its increased absorption into the bloodstream.

In the data illustrated in FIG. 5, the recording of SSP confirmed our previous studies (Kroening et al., 2003) that 10 mM cGMP alone produces, but that 2.8 nM cGMP alone generally does not produce, the SSP waveform. Infusion of cGMP alone, at concentrations below 1 mM is incapable of stimulating SSP and, therefore, does not slow intestinal transit (see Table 1). However, the combination of 2.8 nM cGMP and 12 mM succinate resulted in a statistically significant increase in the SSP number as compared to saline infusion controls and as compared to cGMP or succinate infused separately. The concentrations of 2.8 nM cGMP and 12 mM succinate approximate their respective secreted concentrations released by the tapeworm into tapeworm-conditioned medium.

These observations show that succinate specifically lowers the minimal concentration of cGMP needed to induce SSP and to prolong intestinal transit. That is, the cGMP concentration required to activate SSP response is reduced from about 10 mM to 2.8 nM, and likely a good deal less, a concentration difference of at least 1,000,000 times.

The lack of SSP induction with succinate alone, lactic acid, acetate, and glucosaminic acid, all found in tapeworm secretions, indicates that these compounds are not acting individually as factors inducing altered motility patterns in the rat intestinal smooth muscle but, like succinate, may act to potentiate the effects of cGMP. The failure of high concentrations of succinate alone (100 mM) to induce SSP (data not shown), indicates that succinate cannot act independently to alter intestinal motility at a higher range, as does cGMP. The failure of cAMP alone to stimulate SSP (FIG. 3) indicates that the activation of SSP is specific to cGMP and is not a characteristic common to the entire genus of cyclic nucleotides.

### Example 4

Effect of cGMP on Feed Conversion Ratio

cGMP for use in this Example was manufactured by ICN Biomedicals (Irvine, Calif.), and purchased from Fisher Scientific (Hampton, N.H.). Male Ross x Ross broiler chicks were obtained from Sunnyside Hatchery (Beaver Dam, Wis.).

Standard commercial chick mash was used as a base ration. cGMP with and without succinate was dissolved in water and sprayed as a mist onto the basal diet during continuous mixing. Diets included 10 mg cGMP/kg of feed; 100 mg succinate/kg of feed; and 10 mg cGMP/kg of feed+100 mg succinate/kg of feed. Diets were prepared 1 to 3 days prior to the start of the experiment and stored in individual plastic buckets (1 bucket/replicate).

Chicks were randomly placed into treatment groups with 5 birds per replicate. Chicks were weighed as a group, so each pen of 5 animals represents n=1. Treatments and replicates were randomly assigned (via a random number generator) to pens. Chicks were fed ad libitum and checked daily.

Feed intake and pen weight were recorded weekly. Feed conversion ratios were calculated as grams of feed consumed/grams of weight gain in the animals. Because the birds were raised to 2 to 3 weeks of age, feed conversion ratios could be calculated for each week or combination of weeks. The results are shown in Table 2 for two sets of experiments. Experiment 1 was to run for 3 weeks, but was accidentally disrupted during the third week. (The animals were fed a different feed composition.) Experiment 2 ran the full 3 weeks planned.

TABLE 2

| | | | Feed Conversion |
|---|---|---|---|
| Experiment 1 | Gain | Feed Intake | Ration FCR |
| Control | 1355 ± 205 | 2144 ± 253 | 1.616 ± 0.077 |
| Succinate | 1372 ± 194 | 2127 ± 257 | 1.561 ± 0.073 |
| cGMP | 1548 ± 65 | 2334 ± 144* | 1.535 ± 0.094 |
| cGMP + | 1279 ± 184 | 2065 ± 179 | 1.608 ± 0.099 |
| succinate | | | |
| Experiment 2 | FCR Week 1 | FCR Week 2 | FCR Week 3 |
| Control | 1.802 ± 0.159 | 1.564 ± 0.105 | 1.654 ± 0.100 |
| cGMP | 1.635 ± 0.093** | 1.459 ± 0.058** | 1.580 ± 0.059** |

*p = 0.09 for week 1 of the study and 0.062 for week 2 of the study. The experiment was terminated prematurely after week 2.
**p < 0.05.

### BIBLIOGRAPHY

U.S. Pat. No. 5,977,175 to Lin.

U.S. Pat. No. 5,817,641 to Waldman et al.

Berry et al. 1986. Am. J. Vet. Res. 47: 27-30.

Bråten and Hopkins. 1969. Parasitology. 59: 891-905.

Bulbring and Crema. 1958. Br. J. Pharmacol. 13:444-457.

Budavari et al. (Eds.). 1989. The Merck Index, Merck & Co., Eleventh Edition, p. 422.

Cain et al. 1977. J. of Parasito. 63: 486-491.

Carey et al. 1994. Am. J. Physiol. 267:R156-R163.

Carlson et al. 1978. J. Pharmacol Exp. Ther. 172:367-376.

Castex et al. 1994. Gastroenterology 107:976-984.

Castro. 1989. Parasitol. Today 5:11-19.

Champaneria et al. 1992. Br. J. Pharmacol. 106:693-696.

Coelho et al. 1986. J. Surg. Res. 41: 274-278.

Cook and Roberts. 1991. J. Parasitology 77: 21-25.

Davis et al. 1986. Pharm. Res. 3:208-213.

de Boer et al. 1996. J. Pharmacol. Exp. Ther. 277:852-860.

Dwinell et al. 1994. Am. J. Physiol. 267(5 Pt. 1):G851-G858.

Dwinell et al. 1997. Am. J. Physiol. 273(2 Pt. 1):G480-G485.

Dwinell et al. 1998. J. Parasitol. 84:673-680.

Findlay et al. 1981. J. Immunol. 126: 1728-1730.

Folch, et al. 1957. J. Biol. Chem. 226: 497-509.

Forte. 1999. Regulatory Peptides 81: 25-39.

Foster and Lee. 1996. Parasitology 112: 97-104.

Graf and Sarna. 1996. Am. J. Physiol. 270:G992-G1000.

Grider et al. 1996. Am. J. Physiol. 270:G778-G782.

Hopkins. 1970. Parasitology 60: 255-271.

Husebye et al. 1994. Dig. Dis. and Sciences 39: 946-956.

Insler and Roberts. 1976. Exp. Parasit. 39: 351-357

Ishikawa et al. 1969. J. Bio. Chem. 244:6371-6376.

Jiang et al. 2000. Gastroenterology 119:1267-1275.

Khosla and Davis. 1987. J. Pharm. Pharmacol 39:47-49.

Kiliaan et al. 1998. Am. J. Physiol. 275:G1037-G1044.

Knowles and Oaks. 1979. J. Parasit. 65: 715-731.

Kroening, et al. 2002. J. Parasitol. 88: 227-231

Kroening et al. 2003. J. Parastol. 89:1136-1142

Lin et al. 1997. Dig. Dis. and Sciences 42: 19-25.

Lordal and Hellstrom. 1999. Neurogastroenterol. Motil. 11:1-10.

Lucchelli et al. 2000. Arch. Pharmacol. 362:284-289.

Madara and Dharmsathaphorn. 1985. J. Cell Bio. 101:2124-2133.

Matheis et al. 1980. Am. J. Physiol. 239:G382-G386.

McLean and Coupar. 1996. Eur J. Pharmacol. 312:215-225.

McKay et al. 1991. Exp. Parasitol. 73:15-26.

Mercer et al. 1987. Mol. & Biochem. Parasit. 26: 225-234.

Mettrick. 1971. Exp. Parasit. 29: 386-401.

Mettrick and Podesta. 1982. Int. J. Parasitol. 12: 151-154.

Nash et al. 1987. J. Clinical Investigation 80:1104-1113.

Niwa and Miyazato. 1996. J. Helminthology 70:33-41.

Oaks and Holy. 1994. Exp. Parasit. 79: 292-300.

Ormsbee et al. 1984. J. Pharmacol. Exp. Ther. 231:436-440.

Ovington and Behm. 1997. Memorias do Instituto Oswaldo Cruz 92:93-104.

Palmer et al. 1984. Exp. Parasit. 57: 132-141.

Palmer and Greenwood-Van Meerveld. 2001. J. Parasit. 87: 483-504.

Pappas and Read. 1972a. J. Parasit. 72: 383-391.

Pappas and Read. 1972b. Biological Bulletin 143: 605-616.

Pappas and Read. 1975. Exp. Parasitol. 37: 469-530.

Pappas and Uglem. 1990. Parasitology 101: 455-464.

Parkos. 1997. Bioessays 19:865-873.

Pineiro-Carrero et al. 1991. Am. J. Physiol. 260:G232-239.

Read and Kilejian. 1969. J. Parasit. 55: 574-578.

Ribeiro and Webb. 1983. Int. J. Parasit. 13: 101-106.

Ruckebusch and Fioramonti. 1975. Gastroenterology 68:1500-1508.

Sagarda et al. 1990. Life Sci. 46:1207-1216.

Schreiber et al. 1998. Eur. Neuropsycholpharmacol. 8:297-302.

Scudamore et al. 1995. J. Exp. Med 182: 1871-1881.

Scott et al. 1988. Am. J. Phys.: Gastrointestinal and Liver Physiology 255: G505-G511.

See et al. 1990. Am. J. Physiol. 259:G593-G598.

Szurszewski. 1969. Am. J. Phys.:Gastrointestinal and Liver Physiology 217: 1757-1763.

Trendelenburg. 1917. Naunyn-Schmiedegerg's Arch. Exp. Phath. Pharmak. 81:55-129.

Uglem and Just. 1983. Science 220: 79-81.

Van DerVorst et al. 1990. Annales de Societe Gelge Med. Tropicale 70:113-120.

Weisbrodt et al. 1980. J. Pharmacol. Exp. Ther. 214:333-338.

Wilson and Wiseman. 1954. J. Physiol. 123:116-125.

Wilson and Baldwin. 1999. Microcirculation 6:189-198.

Yamamoto et al. 1999. Neurogastroenterol Motil. 11:457-465.

Zavras and Roberts. 1984. J. Parasitol 70, 937.

Zavras and Roberts. 1985. J. Parasitol. 70: 937-944 (1985).

Zhao et al. 2000. Am. J. Phys.:Gastrointestinal and Liver Physiology 278: G866-870.

What is claimed:

1. A method for prolonging residence time of an administered substance in the small intestine of a subject, the method comprising:
administering to a subject in need of the substance a composition comprising, in combination, cGMP, succinate, and a pharmaceutically suitable carrier, wherein the composition is administered in an amount and form effective to promote contact of the cGMP and succinate with the subject's small intestine, thereby prolonging the residence time of the administered substance in the small intestine of the subject.

2. The method of claim 1, wherein the composition is administered orally.

3. The method of claim 1, further comprising administering the composition concurrently with the substance, administering the composition prior to administering the substance, or administering the composition after administering the substance, or any combination thereof.

4. The method of claim 3, where the administered substance includes one or more of an active pharmaceutical agent, vitamin, supplement, or nutriceutical.

5. The method of claim 1, wherein the cGMP is in solid, semi-solid, or liquid form.

6. The method of claim 1, wherein the cGMP is selected from the group consisting of: cyclic guanosine 3′,5′-cyclic monophosphate; guanosine 3′,5′-monophosphate; 3′,5′-GMP; cGMP; guanosine 3′,5′-(hydrogen phosphate); guanosine 3′,5′-cyclic monophosphate; and guanosine 3′, 5′-cyclic phosphate.

7. The method of claim 1, wherein the pharmaceutically suitable carrier is selected from the group consisting of tablets, capsules, solutions, emulsions, and suspensions.

8. The method of claim 1, whererein the combination of cGMP and succinate are administered in the form of a solution having a cGMP concentration of from about 1 nM to about 100 mM and having a succinate concentration of from about 1 mM to about 100 mM (measured as the concentration of free succinate).

9. A method of enhancing absorption of orally-administered pharmaceuticals, vitamins, and supplements, the method comprising:
administering to a subject a composition comprising, in combination, an absorption-enhancing of amount of cGMP and succinate, disposed in a pharmaceutically suitable carrier therefor.

10. The method of claim 9, wherein the composition is administered orally.

11. The method of claim 9, further comprising administering the composition concurrently with the substance, administering the composition prior to administering the substance, or administering the composition after administering the substance, or any combination thereof.

12. The method of claim 9, wherein the cGMP is in solid, semi-solid, or liquid form.

13. The method of claim 9, wherein the cGMP is selected from the group consisting of cyclic guanosine 3′,5′-cyclic monophosphate; guanosine 3′,5′-monophosphate; 3′,5′-GMP; cGMP; guanosine 3′,5′-(hydrogen phosphate); guanosine 3′,5′-cyclic monophosphate; and guanosine 3′,5′-cyclic phosphate.

14. The method of claim 9, wherein the pharmaceutically suitable carrier is selected from the group consisting of tablets, capsules, solutions, emulsions, and suspensions.

15. The method of claim 1, whererein the combination of cGMP and succinate is administered in the form of a solution having a cGMP concentration of from about 1 nM to about 100 mM and having a succinate concentration of from about 1 mM to about 100 mM (measured as the concentration of free succinate).

16. A method of increasing bioavailability of an orally-ingested pharmaceutical, vitamin, or nutritional supplement, the method comprising:
administering to a subject a composition comprising, in combination, a bioavailability-increasing amount of cGMP and succinate, disposed in a pharmaceutically suitable carrier therefor.

17. A method of improving feed conversion ratios in monogastric animals, the method comprising:
administering to a moniogastric animal a feed composition comprising a base feed ration in combination with an added amount of cGMP, wherein the added amount of cGMP is sufficient to improve the feed conversion ratio of the monogastric animal.

18. The method of claim 17, wherein the feed composition is administered to avians.

19. The method of claim 17, wherein the feed composition is administered to mammals.

20. The method of claim 17, wherein the feed composition is administered to swine.

21. The method of claim 17, wherein the feed composition further comprises an added amount succinate.

22. The method of claim 21, wherein the feed composition is administered to avians.

23. The method of claim 21, wherein the feed composition is administered to mammals.

24. The method of claim 21, wherein the feed composition is administered to swine.

25. A composition for prolonging residence time of an administered substance in the small intestine of a subject, the composition comprising, in combination, a residence time-increasing amount of cGMP and succinate, disposed in a pharmaceutically suitable carrier therefor.

26. The composition of claim 25, wherein the pharmaceutically suitable carrier is a solid, semi-solid, or liquid.

27. The composition of claim 25, wherein the cGMP is selected from the group consisting of cyclic guanosine 3′,5′-cyclic monophosphate; guanosine 3′,5′-monophosphate; 3′,5′-GMP; cGMP; guanosine 3′,5′-(hydrogen phosphate); guanosine 3′,5′-cyclic monophosphate; and guanosine 3′,5′-cyclic phosphate, and the succinate is selected from the group consisting of succinic acid, pharmaceutically suitable mono- and di-salts thereof, and pharmaceutically suitable mono- and di-esters thereof

28. The composition of claim 25, the cGMP and the succinate are disposed in a liquid carrier having a cGMP concentration of from about 1 nM to about 100 mM and having a succinate concentration of from about 1 mM to about 100 mM (measured as the concentration of free succinate).

29. An animal feed composition comprising a base feed ration in combination with an added amount of cGMP, wherein the added amount of cGMP is sufficient to improve feed conversion ratios of monogastric animals fed the composition.

30. An animal feed composition of claim 29, further comprising an added amount of succinate.
